(19) United States
(12) Patent Application Publication
(10) Pub. No.: US 2016/0015844 A1
(43) Pub. Date: Jan. 21, 2016

(54) Sanitizing Floor Mat

(71) Applicant: Juliana Collins, Ontario, CA (US)

(72) Inventor: Juliana Collins, Ontario, CA (US)

(21) Appl. No.: 14/804,767

(22) Filed: Jul. 21, 2015

Related U.S. Application Data
(60) Provisional application No. 62/026,891, filed on Jul. 21, 2014.

(51) Int. Cl. A61L 2/18

(52) U.S. Cl. CPC A61L 2/18

(57) ABSTRACT

A sanitizing floor mat for sanitizing a user's shoes when the user steps thereon in order to prevent the spread of germs and bacteria on the user's shoes. The sanitizing floor mat includes a body that is substantially planar so that it can be placed flush on the floor, wherein the mat is preferably placed in the threshold of a doorway. The body is composed of an absorbent material and is impregnated with a disinfecting solution. The disinfecting solution is preferably an antiseptic solution. A base layer is disposed on an underside of the body, wherein the base layer is composed of a high-friction material so as to prevent the sanitizing floor mat from slipping on the floor.

### CROSS REFERENCE TO RELATED APPLICATION

This application claims the benefit of U.S. Provisional Application No. 62/026,891 filed on Jul. 21, 2014. The above identified patent application is herein incorporated by reference in its entirety to provide continuity of disclosure.

### BACKGROUND OF THE INVENTION

### Field of the Invention

The present invention relates to floor mats. More specifically, the present invention provides a sanitizing floor mat having a body impregnated with a disinfecting solution, such as an antiseptic, in order to sanitize the user's shoes when the user steps thereon.

It is of great importance to prevent the spread of germs and bacteria inside of hospitals and other healthcare facilities. Care is taken to sanitize the rooms of hospitals, the instruments used in treating patients, and the healthcare professionals themselves take precautions to ensure that they remain sterile. Nurses and physicians often scrub or wash their hands and wear gloves in order to prevent spreading germs to the patient or from becoming a carrier of the patient's germs, wherein the physician may then spread germs to other patients. Further, hospital workers may wear scrubs and facemasks, among other disposable garments in order to further reduce spread of germs.

While many precautions are taken to prevent the spread of germs in a healthcare facility, patients and healthcare professionals alike often travel between multiple rooms of the hospital and walk through common areas, such as hallways, reception areas, and waiting rooms. Thus, patients and healthcare professionals may spread germs on their shoes as they walk through the hospital. The floors of a patient room may become unsanitary by having a number of people walk thereover, or if bodily fluids and the like are spilled onto the floor. Accordingly, a sanitizing floor mat is desired in order to quickly and easily sanitize a person's shoes as they travel between various rooms of a hospital.

Devices have been disclosed in the prior art that relate to floor mats having disinfecting properties. These include devices that have been patented and published in patent application publications. These devices generally relate to floor mats for cleaning the user's shoes and removing dirt therefrom, such as U.S. Published Patent Application Number 2012/0177714, U.S. Published Patent Application Number 2012/0167325, U.S. Pat. No. 3,526,015, U.S. Pat. No. 3,696,459, U.S. Published Patent Application Number 2013/0198990, and U.S. Published Patent Application Number 2007/0254015.

These prior art devices have several known drawbacks. The devices in the prior art provide floor mats for use in removing dirt from a user's shoes and include various types of cleaning agents or solutions. The floor mats of the prior art fail to provide a body composed of an absorbent material, such as foam, that is impregnated with a disinfecting solution and that further includes a base layer composed of a high-friction material.

In light of the devices disclosed in the prior art, it is submitted that the present invention substantially diverges in design elements from the prior art and consequently it is clear that there is a need in the art for an improvement to existing sanitizing floor mat devices. In this regard the instant invention substantially fulfills these needs.

### SUMMARY OF THE INVENTION

In view of the foregoing disadvantages inherent in the known types of floor mats now present in the prior art, the present invention provides a new floor mat wherein the same can be utilized for providing convenience for the user when allowing the user to sanitize his or her shoes.

It is therefore an object of the present invention to provide a new and improved sanitizing floor mat comprising a body composed of an absorbent material, wherein the body is impregnated with a disinfecting solution.

It is another object of the present invention to provide a sanitizing floor mat having a body that is substantially planar and rectangular in configuration so that it can be placed on the floor in the threshold of a doorway.

Another object of the present invention is to provide a sanitizing floor mat impregnated with a disinfecting solution comprising an antiseptic solution.

Yet another object of the present invention is to provide a sanitizing floor mat having a base layer on an underside thereof, wherein the base layer is composed of a high-friction material.

Another object of the present invention is to provide a sanitizing floor mat that may be readily fabricated from materials that permit relative economy and are commensurate with durability.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of the sanitizing floor mat of the present invention.

FIG. 2 shows a cross-sectional view of the sanitizing floor mat.

FIG. 3 shows a perspective view of an alternate embodiment of the sanitizing floor mat.

### DETAILED DESCRIPTION OF THE INVENTION

Reference is made herein to the attached drawings. Like reference numerals are used throughout the drawings to depict like or similar elements of the sanitizing floor mat. For the purposes of presenting a brief and clear description of the present invention, the preferred embodiment will be discussed as used for sanitizing a person's shoes. The figures are intended for representative purposes only and should not be considered to be limiting in any respect.

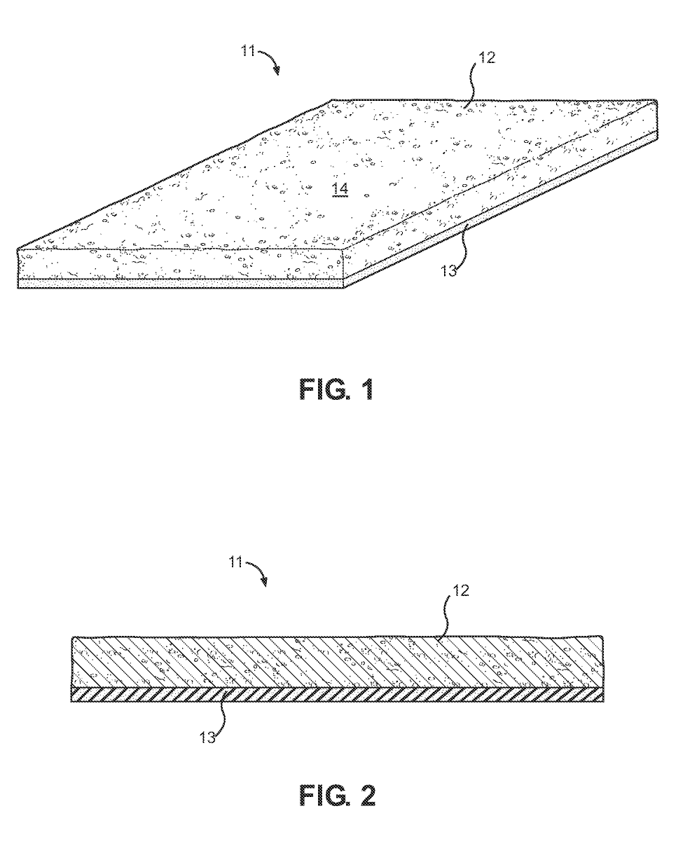

Referring now to FIG. 1, there is shown a perspective view of the sanitizing floor mat of the present invention. The sanitizing floor mat 11 comprises a body 12 that is composed of an absorbent material. The absorbent material is preferably a foam or sponge-like material. The body 12 is shown as having a rectangular configuration, however, in alternate embodiments the body 12 may have any of various other shapes including square or circular shapes, among others. The body 12 is substantially planar so that it can rest flush against the surface of a floor, and can contour to any curvature or imperfections on the surface of the floor. In a preferred embodiment, the body 12 is substantially rectangular so as to fit within the threshold of a doorway so as to help ensure that a person steps on the floor mat 11 when exiting or entering a room. In this way, as a person enters a room, the user steps on the floor mat 11 sanitizing his or her shoes prior to entering the room so as to prevent germs from being introduced into the room. Similarly, the person must step on the floor mat 11 when exiting the room, in order to prevent any germs within the room from being transported out of the room on the user's shoes.

The body 12 is impregnated with a disinfecting solution. The disinfecting solution is preferably an antiseptic solution so as to help to kill any germs or bacteria on the user's shoes. In some embodiments, the disinfecting solution may also include bleach or a similar cleaning agent. Thus, the disinfecting solution is adapted to sanitize the user's shoes, particularly the soles thereof, when the user steps onto the body 12, causing the impregnated disinfecting solution to come into contact with the user's shoes. The body 12 contains sufficient disinfecting solution so that disinfecting solution is released from the body and contacts the user's shoes without making the user's shoes wet and slippery, which may be potentially dangerous or harmful.

Referring now to FIG. 2, there is shown a cross-sectional view of the sanitizing floor mat of the present invention. The sanitizing floor mat 11 preferably comprises a base layer 13 disposed on the underside of the body 12. The base layer 13 is composed of a high-friction material so as to prevent the sanitizing floor mat 11 from slipping on the surface on which it is placed. The base layer 13 is preferably composed of rubber or silicone, however, in alternate embodiments, other high-friction materials may be used. The base layer 13 is also water impermeable so as to prevent any disinfecting solution impregnated in the body 12 from passing through the underside of the floor mat 11 and contacting the ground, which may cause the floor to become wet or slippery.

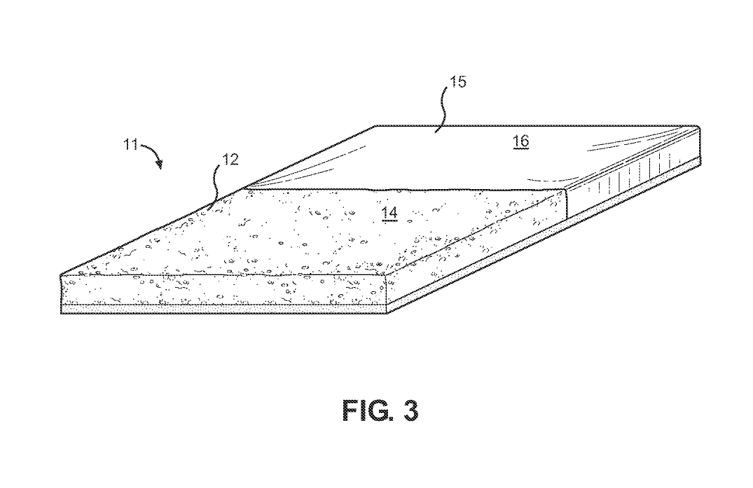

Referring now to FIG. 3, there is shown a perspective view of an alternate embodiment of the sanitizing floor mat. In the illustrated embodiment, the floor mat 11 comprises a body 12 having a drying section 15 in addition to the absorbent section. The absorbent section is composed of an absorbent material that is impregnated with a disinfecting solution, as described above. The body also 12 also comprises a drying section 15 thereon having a flat upper surface 16. The drying section 15 is adapted to dry the user's shoes, and may be composed of a fabric material. Once the user steps on the absorbent section of the floor mat 11 so that disinfecting solution therein is released onto the user's shoes, the user may then step onto the drying section 15 in order to remove any excess disinfecting solution so that the user's shoes do not become wet or slippery. The drying section 15 is integral to the body 12 of the floor mat 11 so that the floor mat 11 retains the rectangular configuration.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A sanitizing floor mat, comprising:
a body comprising an absorbent material;
said body impregnated with a disinfecting solution;
a base layer disposed on an underside of said body, wherein said base layer is composed of a high-friction material.

2) The sanitizing floor mat of claim 1, wherein said body is composed of foam.

3) The sanitizing floor mat of claim 1, wherein said disinfecting solution comprises an antiseptic solution.

4) The sanitizing floor mat of claim 1, wherein said disinfecting solution comprises bleach.

5) The sanitizing floor mat of claim 1, wherein said body is substantially rectangular in configuration.

6) The sanitizing floor mat of claim 1, wherein said body is substantially planar.

7) The sanitizing floor mat of claim 1, wherein said base layer is composed of rubber.

8) The sanitizing floor mat of claim 1, wherein said base layer is composed of a waterproof material.

9) A sanitizing floor mat, comprising:
a body having an absorbent section and a drying section;
wherein said absorbent section is composed of an absorbent material;
wherein said absorbent section is impregnated with a disinfecting solution;
a base layer disposed on an underside of said body, wherein said base layer is composed of a high-friction material.

10) The sanitizing floor mat of claim 9, wherein said body is substantially rectangular in configuration.
